(19) United States
(12) Patent Application Publication
(10) Pub. No.: US 2024/0237918 A1
(43) Pub. Date: Jul. 18, 2024

(54) SYSTEM AND METHOD FOR DETERMINING A BODY COMPOSITION IN A HYDROSTATIC ENVIRONMENT

(71) Applicant: Austin Bond, The Colony, TX (US)

(72) Inventor: Austin Bond, The Colony, TX (US)

(21) Appl. No.: 18/413,216

(22) Filed: Jan. 16, 2024

Related U.S. Application Data
(60) Provisional application No. 63/480,114, filed on Jan. 17, 2023.

(51) Int. Cl. A61B 5/107; A61B 5/00; G01G 19/44; G16H 40/63

(52) U.S. Cl. CPC A61B 5/1073; A61B 5/7278; A61B 5/741; A61B 5/749; G01G 19/44; G16H 40/63

(57) ABSTRACT

A system and method for determining body composition of subject in hydrostatic environment is disclosed. The system includes weighing device to measure dry weight and wet weight of subject, subject load transfer structure to receive load of subject, container with liquid, and container load transfer structure to transfer load from weighing device to container. System includes processors to receive user input comprising attributes of subject and determine residual lung volume of subject. Further, processors determine body density based on residual lung volume, gastrointestinal volume, water density, and both dry and wet weights of subject (shown in equation (3)). Further, processors determine fat-mass fraction and lean mass fraction of subject based on body composition derived from body density.

### CROSS REFERENCE TO RELATED APPLICATION(S)

This application claims the priority to incorporates by reference the entire disclosure of U.S. provisional patent application No. 63/480,114, filed on Jan. 17, 2023, and titled “SYSTEM AND METHOD FOR IMPROVING HYDROSTATIC BODY COMPOSITION MEASUREMENT (HBCM)”.

### TECHNICAL FIELD

Embodiments of the present disclosure generally relate to body composition measurement systems and more particularly relate to a system and a method for determining a body composition in a hydrostatic environment.

### BACKGROUND

Generally, in scientific, medical, and fitness communities, there is growing concern over the obesity epidemic. Unfortunately, individuals seeking to manage their weight often rely on subjective methods, such as personal experience or biased recommendations from coaches, physicians, and researchers, without any reliable way of objectively measuring progress. This approach is flawed because controlling a system parameter, such as body composition, requires an accurate measurement of the system's state. Many people approach weight loss in an unscientific manner, which can be unreliable, particularly for those with pathological conditions. Thus, a scientific measurement of body composition is necessary to provide reliable feedback.

Currently, it is essential to understand the basic principles of feedback control systems to manage weight effectively. Sensory input is required as a feedback mechanism when an individual seeks to control a system parameter, such as body composition. Without measuring the system parameters, it is like driving a car while blindfolded and expecting not to run off the road. Some people rely on an “artistic” approach to weight loss, making changes based on how they feel. However, this approach is unreliable, especially for those with underlying and often undiagnosed or untreated pathological conditions that affect their visceral body fat content. A scientific measurement of body composition is necessary to retrain intuition accurately.

Conventionally, many people debate the accuracy of different body composition testing methods. Two common traps that individuals seeking to educate themselves regarding body composition testing fall into are assuming that consumer products on the market, such as electrical impedance scales and skin calipers, are accurate and forgetting that these methods are calibrated against another method, which is usually hydrostatic body composition measurement (HBCM)—hence why HCBM is often referred to as the “Gold Standard”. These traps can lead to misleading results. Conventional, commercial off-the-shelf systems, such as electrical-impedance scales, produce measurements that fluctuate widely due to changes in hydration, metabolic state, and inflammatory activity. These fluctuations result in a low signal-to-noise ratio, rendering the data useless. Another conventional system, the skin caliper, is only useful for those with relatively low body fat levels that can be pinched easily, such as well-trained athletes. The method is unable to accurately measure the visceral body fat content of individuals with underlying pathological conditions that cannot be pinched easily. Yet another conventional method utilizes dual-energy x-ray absorption (DEXA) method for multiple modality body composition analysis. However, the DEXA) method is high cost and limited availability and restricted to laboratory and hospital environments. Some individuals may choose to mount the conventional system DEXA on a mobile platform such as truck or van to increase access. However, there are measurement risks and hazards presented when the vehicle is subjected to transportation shock and vibration loads that exceed manufacturer specifications. Usually, companies that manufacture biomedical devices do not provide due diligence to design, analyze, and test against the full range of transportation loads. Oversight on transportation loads can compromise the functionality and accuracy of the instrument.

Therefore, there is a need for an improved system and method for determining a body composition in a hydrostatic environment to address at least the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

An aspect of the present disclosure provides a system for determining a body composition in a hydrostatic environment. The system comprises a weighing device configured to measure at least one of a dry weight, and a wet weight corresponding to a body density of the subject. Further, the system comprises a subject load transfer structure, comprising a horizontal platform and a plurality of structural elements coupled to the horizontal platform, configured to receive a load of a subject for determining the wet weight of the subject. Furthermore, the system comprises a container, comprising a liquid and the subject load transfer structure within the liquid, for determining the wet weight of the subject in a hydrostatic environment, when the subject is submerged in a pre-defined posture in the liquid. Furthermore, the system comprises a container load transfer structure, coupled to the weighing device, configured to transfer the load from the weighing device to the container. Additionally, the system comprises one or more hardware processors communicatively connected to the weighing device and a memory coupled to the one or more hardware processors. The one or more hardware processors receive, through a display associated with the one or more hardware processors, a user input comprising one or more attributes of the subject. Further, the one or more hardware processors determine a residual lung volume of the subject based on the received one or more attributes. Furthermore, the one or more hardware processors determine the body density of the subject based on the determined residual lung volume, gastrointestinal volume, water density, and both dry and wet weights (shown in equation (8)). Additionally, the one or more hardware processors determine the body composition of the subject based on the determined body density of the subject. Furthermore, the one or more hardware processors determine a fat mass and a fat-free mass of the subject based on a correlation between a fat mass fraction and the determined body density. Additionally, the one or more hardware processors output on the display the body composition of the subject, based on the determined fat mass and the fat-free mass of the subject.

An aspect of the present disclosure provides a method for determining a body composition in a hydrostatic environment. The method includes receiving, through a display associated with the one or more hardware processors, a user input comprising one or more attributes of a subject. Further, the method includes determining at least one of a residual lung volume of the subject based on the received one or more attributes. Furthermore, the method includes determining a body density of the subject based on the determined residual lung volume, gastrointestinal volume, water density, a dry weight, and a wet weight, of the subject (shown in equation (8)). Furthermore, the method includes determining a fat mass and a fat-free mass of the subject based on a correlation between a fat mass fraction and the determined body density. Additionally, the method includes outputting, on the display, the body composition of the subject, based on the determined fat mass and the fat-free mass of the subject.

Yet another aspect of the present disclosure provides a non-transitory computer-readable storage medium having programmable instructions stored therein. That when executed by one or more hardware processors, cause the one or more hardware processors to receive, through a display associated with the one or more hardware processors, a user input comprising one or more attributes of a subject. The one or more hardware processors determine a residual lung volume of the subject based on the received one or more attributes. Furthermore, the one or more hardware processors determine a body density of the subject based on the determined residual lung volume, gastrointestinal volume, water density, and a dry weight, and a wet weight, of the subject (shown in equation (3)). Furthermore, the one or more hardware processors determine a fat mass and a fat-free mass of the subject based on a correlation between a fat mass fraction and the determined body density. Additionally, the one or more hardware processors output, on the display, the body composition of the subject, based on the determined fat mass and the fat-free mass of the subject.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF ACCOMPANYING DRAWINGS

The disclosure will be described and explained with additional specificity and detail with the accompanying figures in which:

FIG. 1 illustrates an exemplary block diagram representation of a network architecture implementing a system for determining a body composition in a hydrostatic environment, in accordance with an embodiment of the present disclosure;

FIG. 2 illustrates an exemplary block diagram representation of a computer-implemented system, such as those shown in FIG. 1, capable of determining the body composition in the hydrostatic environment, in accordance with an embodiment of the present disclosure;

FIG. 3 illustrates an exemplary schematic diagram representation of the system, such as those shown in FIG. 1, for determining the body composition in the hydrostatic environment, in accordance with an embodiment of the present disclosure;

FIG. 4 illustrates a flow chart depicting a method for determining the body composition in the hydrostatic environment, according to an example embodiment of the present disclosure;

FIG. 5 illustrates an exemplary block diagram representation of a hardware platform for an implementation of the disclosed system, according to an example embodiment of the present disclosure;

FIG. 6 is an exemplary view depicting an infrared camera image of the subject during a testing procedure, in accordance with an embodiment of the present disclosure; and

FIG. 7 is a graphical representation depicting a typical plot of wet weigh scale output during the testing procedure, such as shown in FIG. 6, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client, or server computer system) configured by an application may constitute a “module” (or “subsystem”) that is configured and operated to perform certain operations. In one embodiment, the “module” or “subsystem” may be implemented mechanically or electronically, so a module includes dedicated circuitry or logic that is permanently configured (within a special-purpose processor) to perform certain operations. In another embodiment, a “module” or s “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired), or temporarily configured (programmed) to operate in a certain manner and/or to perform certain operations described herein.

Embodiments of the present disclosure provide a system and a method for determining a body composition in a hydrostatic environment. The present disclosure provides accurate results, by using underwater weighing with the correlation method developed from human cadavers for measuring the user's fat mass and fat-free mass. The present disclosure provides point-to-point trend tracking in body fat, which is important for people who are trying to lose or gain weight. The present disclosure eliminates the use of swinging plates, and requires minimal equipment, making it more cost-effective. The present disclosure eliminates settling time of the swinging plate, which is uncomfortable for the user. In contrast, the present disclosure provides quick measurements, reducing discomfort and the risk of inhaling water. The present disclosure reduces the risk of error by assuming residual lung volume (as shown in Equation (1)), based on height and age correlations, which are effective predictors of these parameters. Additionally, residual lung volume must be accounted for, which eliminates the risk of error in the results. The present disclosure ensures accuracy by leveling the container (e.g., water tank) mounted on a platform. The present disclosure eliminates the need for a technician to read the scale, which allows users to take measurements independently and in private. This feature may appeal to individuals who prefer not to have their body composition measurements taken in a public setting. Further, the present disclosure reduces the potential for human error and saves time for both the user and technician, by checking the user's form in the container.

The hydrostatic body composition measurement or a body composition determination may also be referred to as underwater weighing or hydro densitometry. All these terms should be considered as synonymous.

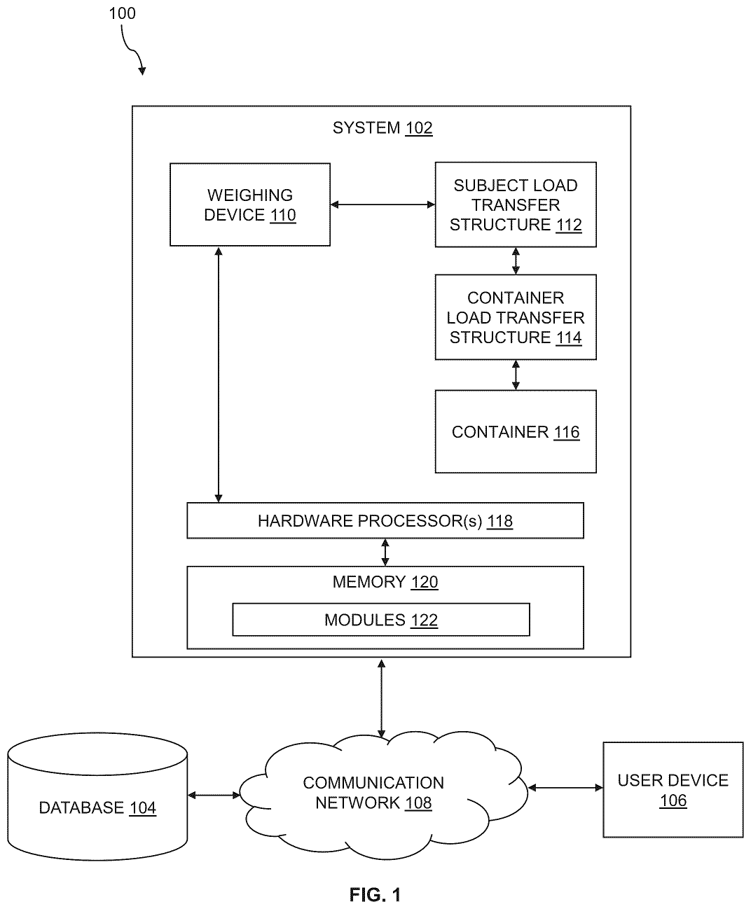

Referring now to the drawings, and more particularly to FIG. 1 through FIG. 5, where similar reference characters denote corresponding features consistently throughout the figures, there are shown preferred embodiments and these embodiments are described in the context of the following exemplary system and/or method.

FIG. 1 illustrates an exemplary block diagram representation of a network architecture implementing a system for determining a body composition in a hydrostatic environment, in accordance with an embodiment of the present disclosure. According to FIG. 1, the network architecture 100 may include the system 102, a database 104, and a user device 106. The system 102 may be communicatively coupled to the database 104, and the user device 106 via a communication network 108. The communication network 108 may be a wired communication network and/or a wireless communication network. The database 104 may include, but is not limited to, reports, load data over a period, subject data, body composition data, any other data, and combinations thereof. The database 104 may be any kind of database such as, but are not limited to, relational databases, dynamic databases, monetized databases, scalable databases, cloud databases, distributed databases, any other databases, and combination thereof.

Further, the user device 106 may be associated with, but not limited to, a user, a subject, an individual, an administrator, a vendor, a technician, a health care worker, a weight loss specialist, instructor, a caretaker, a patient, a supervisor, a team, an entity, a facility, and the like. The user device 106 may be used to provide input and/or receive output to/from the system 102, and/or to the database 104, respectively. The user device 106 may present to the user one or more user interfaces for the user to interact with the system 102 and/or to the database 104 for the body composition determining needs. The user device 106 may be at least one of, an electrical, an electronic, an electromechanical, and a computing device. The user device 106 may include, but is not limited to, a mobile device, a smartphone, a Personal Digital Assistant (PDA), a tablet computer, a phablet computer, a wearable computing device, a Virtual Reality/Augmented Reality (VR/AR) device, a laptop, a desktop, a server, and the like. The entities and the facility may include, but are not limited to, a hospital, a healthcare facility, an exercise facility, a laboratory facility, an e-commerce company, a merchant organization, an airline company, a hotel booking company, a company, an outlet, a manufacturing unit, an enterprise, an organization, an educational institution, a secured facility, a warehouse facility, a supply chain facility, any other facility and the like.

Further, the system 102 may be implemented by way of a single device or a combination of multiple devices that may be operatively connected or networked together. The system 102 may be implemented in hardware or a suitable combination of hardware and software. The system 102 includes a weighing device 110, a subject load transfer structure 112, a container load transfer structure 114, a container 116, one or more hardware processor(s) 118, and a memory 120. The memory 120 may include a plurality of modules 122. The system 102 may be a hardware device including the one or more hardware processor(s) 118 executing machine-readable program instructions for determining the body composition in the hydrostatic environment. Execution of the machine-readable program instructions by the one or more hardware processor(s) may enable the proposed system 102 to determine the body composition in the hydrostatic environment. The “one or more hardware processor(s) 118” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The one or more hardware processor(s) 118 may include, for example, microprocessors, microcomputers, microcontrollers, digital signal processors, central processing units, state machines, logic circuits, and/or any devices that manipulate data or signals based on operational instructions. Among other capabilities, the one or more hardware processor(s) 118 may fetch and execute computer-readable instructions in the memory 120 operationally coupled with the system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, assets, machinery, instruments, facility equipment, life safety devices, intensive care devices, treatment devices, emergency management devices, health care devices, laboratory devices, testing kits, any other devices, weighing machine, image capturing devices, and combination thereof. The person skilled in the art should not be limiting the components/subsystems shown in FIG. 1. Although FIG. 1 illustrates the system 102, and the user device 106 connected to the database 104, one skilled in the art can envision that the system 102, and the user device 106 can be connected to several user devices located at different locations and several databases via the communication network 108.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices that were known in the art.

In an exemplary embodiment, the system 102 may execute the weighing device 110 to measure a dry weight corresponding to a body density of a subject (not shown in FIG. 1), and/or a wet weight corresponding to the body density of the subject. The subject may include, but not limited to, a human, an animal, a plant, an object, a substance, a microorganism, geological samples, any other subject, and combination thereof.

In an exemplary embodiment, the system 102 may include the subject load transfer structure 112. The subject load transfer structure 112 comprises a horizontal platform (not shown in FIG. 1) and a plurality of structural elements (not shown in FIG. 1) coupled to the horizontal platform. In an exemplary embodiment, the subject load transfer structure 112 may receive a load of the subject for determining the wet weight of the subject. In an exemplary embodiment, the plurality of structural elements is coupled to the weighing device 110 through a weighing device interface plate (not shown in FIG. 1). In an exemplary embodiment, the weighing device interface plate may transfer the load of the subject from the plurality of structural elements to the weighing device 110.

In an exemplary embodiment, the system 102 may include the container 116. The container 116 may comprise a liquid and the subject load transfer structure 112 within the liquid. The container 116 may be used for determining the wet weight of the subject in the hydrostatic environment, when the subject is submerged in a pre-defined posture in the liquid. In an exemplary embodiment, the container 116 may include, but is not limited to, a plastic container, a glass container, a metal container, a ceramic container, a flexible container, a stainless steel container, a vacuum-sealed container, tanks, barrels, any other containers capable of holding the liquid, and a combination thereof. Further, the liquid comprises, but is not limited to, water, saltwater, oil, ethylene glycol, glycerine, glycerol, ethanol, silicone oil, hydrogen peroxide, fluorocarbon liquid, any other liquids, and combination thereof.

In an exemplary embodiment, the system 102 may include the container load transfer structure 114. The container load transfer structure 114 may be coupled to the weighing device 110. The container load transfer structure 114 may transfer the load from the weighing device 110 to the container 116.

In an exemplary embodiment, the system 102 may include the one or more hardware processors 118 communicatively connected to the weighing device 110. In an exemplary embodiment, the system 102 may execute the one or more hardware processors 118 to receive, through a display (not shown in FIG. 1) associated with the one or more hardware processors 118, a user input comprising one or more attributes of the subject. In an exemplary embodiment, the one or more attributes includes, but not limited to, a height, an age, a gender, a body measurements (e.g. waist circumference, hip circumference), a level of physical activity, medical history (e.g. presence of certain medical conditions), dietary habits (e.g. intake of carbohydrates, protein, and fat), any other attributes, and combination thereof.

In an exemplary embodiment, the system 102 may execute the one or more hardware processors 118 to determine a residual lung volume of the subject based on the received one or more attributes.

In an exemplary embodiment, the system 102 executes the one or more hardware processors 118 to determine the body density of the subject based on at least one of the determined residual lung volume, gastrointestinal volume, water density, and both dry and wet weights (shown in below equation (3)). In an exemplary embodiment, the system 102 executes the one or more hardware processors 118 to determine the body composition of the subject based on the estimated residual lung volume, gastrointestinal volume, water density, and both of the measured dry and wet weights (shown in below equation (3)). In an exemplary embodiment, the system 102 executes the one or more hardware processors 118 to determine a fat mass and a fat-free mass of the subject based on a correlation between a fat mass fraction and the determined body density. The fat mass fraction is the percentage of body weight that is made up of fat tissue. It is used as an indicator of body composition and health status. The fat mass fraction can be calculated by dividing the total fat mass by the total body mass. For example, if a person weighs 70 kg and has a fat mass of 21 kg, their fat mass fraction would be 30% (21 kg/70 kg×100). A healthy fat mass fraction can vary depending on factors such as age, sex, and fitness level, but generally, a fat mass fraction of less than 25% is considered healthy for men and less than 30% is considered healthy for women. Excess fat mass fraction has been associated with a range of health issues, including diabetes, heart disease, and certain types of cancer. In an exemplary embodiment, the system 102 execute the one or more hardware processors 118 to output on the display, a body composition of the subject, based on the determined fat mass and the fat-free mass (i.e., lean mass fraction) of the subject.

In an exemplary embodiment, the system 102 further comprises an image capturing device (not shown in FIG. 1). The image capturing device may be configured to monitor the subject during the measurement of the wet weight, using one or more image processing based artificial intelligence (AI) techniques. In an exemplary embodiment, monitoring the subject comprises determining if the subject is submerged in the pre-defined posture in the liquid.

In an exemplary embodiment, the system 102 further comprises a speaker device (not shown in FIG. 1). The speaker device may be configured to output an audio corresponding to, but is not limited to, a beginning of the measurement of at least one of the dry weight and the wet weight, a completion of the measurement of at least one of the dry weight and the wet weight, a formation instruction of the pre-defined posture, a breath hold instruction for an empty-lung breath hold of the subject, and the like. The wet weight of the subject is measured based on the empty-lung breath hold of the subject to reduce the lungs of the subject to the residual lung volume.

In an exemplary embodiment, the system 102 further comprises a microphone (not shown in FIG. 1). The microphone may be configured to receive an utterance from at least one of the subject and one or more users, for a remote support corresponding to the measurement (e.g. a two-way radio/“walkie-talkie”) or an intercom system).

In an exemplary embodiment, the system 102 further comprises a levelling member (not shown in FIG. 1). The levelling member comprises one or more limit switches and coupled to the container 116. The levelling member may be configured to detect at least one of a level of the container 116, an attitude of the container 116, and a contact status of the container 116.

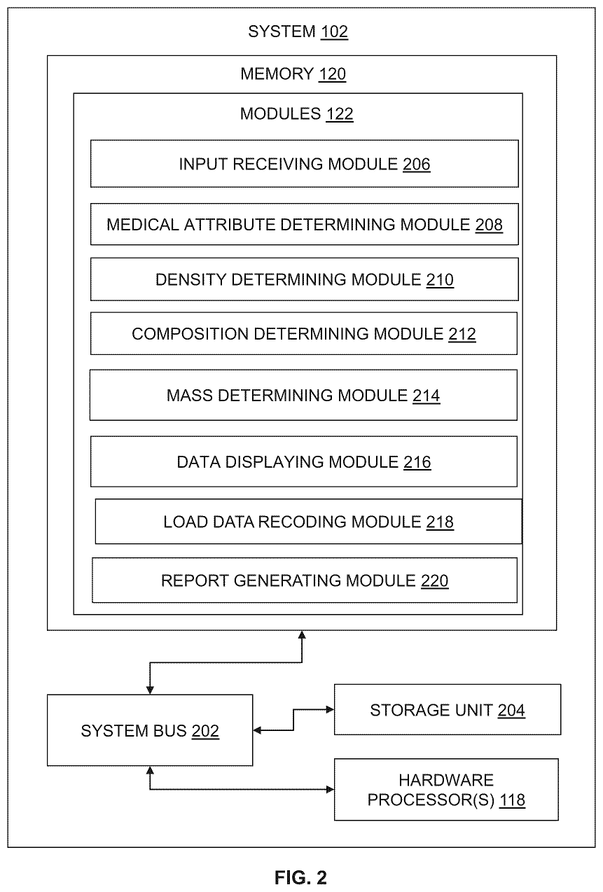

FIG. 2 illustrates an exemplary block diagram representation of a computer-implemented system 102, such as those shown in FIG. 1, capable of determining the body composition in the hydrostatic environment, in accordance with an embodiment of the present disclosure. The system 102 may also function as a computer-implemented system (hereinafter referred to as the system 102). The system 102 comprises the one or more hardware processors 118, the memory 120, and a storage unit 204. The one or more hardware processors 118, the memory 120, and the storage unit 204 are communicatively coupled through a system bus 202 or any similar mechanism. The memory 120 comprises a plurality of modules 122 in the form of programmable instructions executable by the one or more hardware processors 118.

Further, the plurality of modules 122 includes an input receiving module 206, a medical attribute determining module 208, a density determining module 210, a composition determining module 212, a mass determining module 214, a data displaying module 216, a load data recording module 218, and a report generating module 220.

The one or more hardware processors 118, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 118 may also include embedded controllers, such as generic or programmable logic devices or arrays, application-specific integrated circuits, single-chip computers, and the like.

The memory 120 may be a non-transitory volatile memory and a non-volatile memory. The memory 120 may be coupled to communicate with the one or more hardware processors 118, such as being a computer-readable storage medium. The one or more hardware processors 118 may execute machine-readable instructions and/or source code stored in the memory 120. A variety of machine-readable instructions may be stored in and accessed from the memory 120. The memory 120 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory 120 includes the plurality of modules 120 stored in the form of machine-readable instructions on any of the above-mentioned storage media and may be in communication with and executed by the one or more hardware processors 118.

The storage unit 204 may be a cloud storage or a database such as those shown in FIG. 1. The storage unit 204 may store, but is not limited to, reports, load data over a period, subject data, body composition data, any other data, and combinations thereof. The storage unit 204 may be any kind of database such as, but are not limited to, relational databases, dynamic databases, monetized databases, scalable databases, cloud databases, distributed databases, any other databases, and combination thereof.

In an exemplary embodiment, the input receiving module 206 may receive, through a display (not shown in FIG. 2) associated with the one or more hardware processors 118, a user input comprising the one or more attributes of the subject. In an exemplary embodiment, the one or more attributes includes, but not limited to, the height, the age, the gender, the body measurements (e.g. the waist circumference, the hip circumference), the level of physical activity, the medical history (e.g. the presence of certain medical conditions), the dietary habits (e.g. intake of carbohydrates, protein, and fat), any other attributes, and combination thereof.

In an exemplary embodiment, the medical attribute determining module 208 may determine the residual lung volume of the subject based on the received one or more attributes. The determination/calculation of the residual lung volume is given below.

$$RLV = Residual Lung Volume , [ L ] \qquad Equation ( 1 )$$

$$A = Age , [ Years ]$$

$$H = Height , [ cm ]$$

$$RLV = ( \frac{11}{400} * A ) + ( 0.0189 * H ) - 2.6139$$

In an exemplary embodiment, the density determining module 210 may determine the body density of the subject based on the estimated residual lung volume, the gastrointestinal volume, the water density (as a function of temperature), and both of the measured dry and wet weights (shown in the equation below).

The determination/calculation of the water density (as the function of temperature) is given below.

$${ρ}_{{H}_{2} O} = mass density of water , [ \frac{Kg}{L} ] \qquad Equation ( 2 )$$

$$T = water temperature , [ ° C . ]$$

$${ρ}_{{H}_{2} O} = ( 1 - ( \frac{T + 288.9414}{508929.2 + 68.12963} ) * {( T - 3.9863 )}^{2} )$$

In an exemplary embodiment, the composition determining module 212 may determine the body composition of the subject based on the determined body density given below.

$${V}_{body} = body volume , [ L ] \qquad Equation ( 3 )$$

$${ρ}_{body} = body density , [ \frac{Kg}{L} ]$$

$${ρ}_{{H}_{2} O} = mass density of water , [ \frac{Kg}{L} ]$$

$${m}_{dry} = dry weight , [ Kg ] , i . e . , unsubmerged weight of test subject$$

$${m}_{wet} = wet weight , [ Kg ] , i . e . , submerged weight of test subject$$

$$RLV = Residual Lung Volume , [ L ]$$

$$GV = Gastrointestinal Volume , [ L ]$$

$${ρ}_{body} = ( \frac{{m}_{dry}}{{V}_{body}} ) = ( \frac{{m}_{dry}}{( \frac{{m}_{dry} - {m}_{wet}}{{ρ}_{{H}_{2} O}} ) - ( RLV + GV )} )$$

In an exemplary embodiment, the mass determining module 214 may determine the fat mass and the fat-free mass of the subject based on the correlation between the fat mass fraction and the determined body density. The fat mass fraction is the percentage of body weight that is made up of fat tissue. It is used as an indicator of body composition and health status. The fat mass fraction can be calculated by dividing the total fat mass by the total body mass. For example, if a person weighs 70 kg and has a fat mass of 21 kg, their fat mass fraction would be 30% (21 kg/70 kg×100). A healthy fat mass fraction can vary depending on factors such as age, sex, and fitness level, but generally, a fat mass fraction of less than 25% is considered healthy for men and less than 30% is considered healthy for women. Excess fat mass fraction has been associated with a range of health issues, including diabetes, heart disease, and certain types of cancer. In an exemplary embodiment, the data displaying module 216 may output on the display, a body composition of the subject, based on the determined fat mass and the fat-free mass (i.e., the lean mass fraction) of the subject.

The determination/calculation of the fat mass fraction is given below.

$$PctFat = body fat percentage , [ \% ] \qquad Equation ( 4 )$$

$$PctFat = ( \frac{457}{{ρ}_{body}} ) - 414.2$$

The determination/calculation of the lean mass fraction is given below.

$$PctLean = Lean mass percentage , [ \% ] \qquad Equation ( 5 )$$

$$PctLean = 100 - PctFat$$

In an exemplary embodiment, the plurality of modules 122 further includes the load data recoding module 218. The load data recoding module 218 may be configured to record, for a pre-defined period, load data corresponding to the load of the subject, from the weighing device 110.

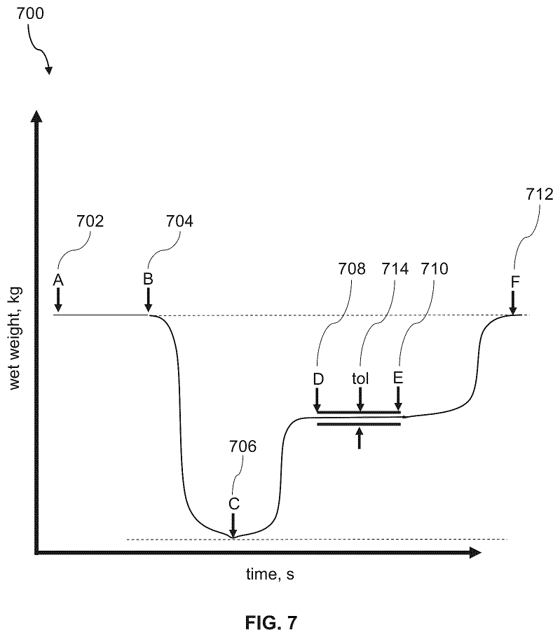

In an exemplary embodiment, the plurality of modules 122 further includes the report generating module 220. Further, the report generating module 222 may be configured to generate one or more reports based on the recorded load data, for the pre-defined period. The one or more reports may include high-level summary data including at least one of the Fat Mass and the Lean Mass Fractions (as shown in Equation (4)), as well as plotted time-series data from the Wet Weight measurement (as shown in FIG. 7).

In an exemplary embodiment, the plurality of modules 122 further includes the data displaying module 216. The data displaying module 216 may be configured to display on the display, the generated one or more reports for inspection, by at least one of the subject and one or more users, of a steady-state value of the body composition.

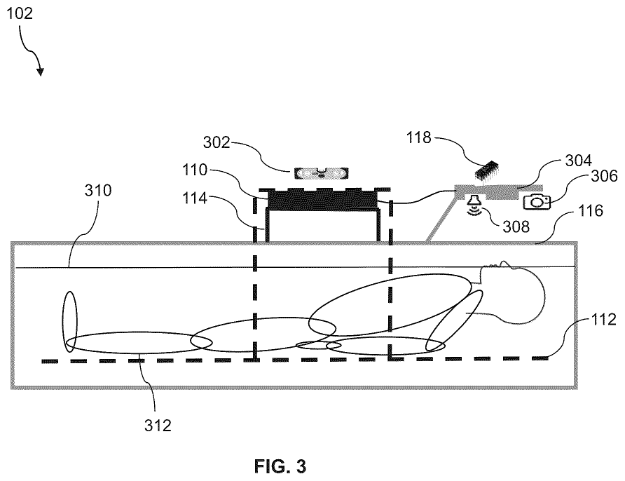

FIG. 3 illustrates an exemplary schematic diagram representation of the system 102, such as those shown in FIG. 1, for determining the body composition in the hydrostatic environment, in accordance with an embodiment of the present disclosure.

The system 102 includes the weighing device 110, the subject load transfer structure 112, the container load transfer structure 114, the container 116, the one or more hardware processor(s) 118, the weighing device interface plate 302, the display 304, the image capturing device 306, the speaker device 308, the microphone (not shown in FIG. 3), and the liquid 310.

The subject 312 may immerse themselves completely inside the liquid 310 beneath a level mark inside the container 116. The subject 312 may rest on the subject load transfer structure 112. The subject load transfer structure 112 resists pendulum motion (i.e., swinging), thereby providing higher precision measurements. The subject load transfer structure 112 comprises of a large, submersed frame which accepts the load from the human test subject referred to the subject 312. The submersed subject load transfer structure 112 transfers the load to the the weighing device 110 via a plurality of structural elements and a horizontal platform which rest on top of the weighing device 110. This configuration is resistant to pendulum motion. A plurality of the conventional systems may employ the use of a swinging plate style of measurement. This is to keep the load-sensing device, either hanging scale, or electro-mechanical load cells out of the water. One primary issue with conventional systems is that the dynamic settling of the swinging plate is relatively slow. This is problematic because the subjects 312 are holding their breath under water while the plate settles. Longer settling times increase the discomfort of the subjects 312 and leads to the potential to inhale water. Individuals with extensive measurement experience have identified an instinctive response for the body to resist exhaling all the air out of the lungs while submerged. To avoid inhale water, exhale all the air out of the lungs while submerged, the present disclosure suggests the subject 312 to arch the upper spine backwards and tilt the chin up such that only the lips and mouth are above the surface of the water as shown in FIG. 3. This allows for a satisfactory achievement of residual lung volume without the risk of inhaling water. It is noted that the industry operators have identified a natural human instinct to not exhale all of the air out of one's lungs while under water. This tendency is mitigated by keeping the mouth above the water, thereby eliminating the feeling of potential drowning. Furthermore, if the vehicle/platform that the tank of water is resting on is unlevel the swinging plate could potentially contact the edge of the tank wall, which may compromise the accuracy. Thus, the platform that the container 116 is mounted to, needs to be levelled.

Consider a scenario of measuring position of the subject 312 within the container 116. The subject 312 is positioned fully beneath the level indicator except for the chin and lips as shown in FIG. 3. Individuals with extensive measurement experience have identified an instinctive response for the body to resist exhaling all the air out of the lungs while submerged. The best practice that the subject matter experts have devised to avoid this scenario is to arch the upper spine backwards and tilt the chin up such that only the lips and mouth are above the surface of the water as shown in FIG. 3. This allows for a satisfactory achievement of residual lung volume without the risk of inhaling water. It is noted that the industry operators have identified a natural human instinct to not exhale all of the air out of one's lungs while under water. This tendency is mitigated by keeping the mouth above the water, thereby eliminating the feeling of potential drowning.

Consider a scenario of determining, by the system 102, a hydrostatic body fat also referred to as underwater weighing. The system 102 receives the height and the age of the subject 312 as input. Further, the system 102 determines the residual lung volume of the subject 312 based on the received height and the age. The height and age are the strongest predictors of residual lung volume corresponding to the subject 312. Conventional systems may measure bone density through ultrasound, x-ray methods, and the like. However, this is usually not necessary since the end user is most interested in point-to-point trends of the body composition or the fat, during which the bone density does not significantly change. Therefore, determining the accurate bone density appears to be needless. However, the residual lung volume must be accurate during the measurement of the hydrostatic body composition measurement of the subject 312.

In case the subject 312 does not exhale all the air out of their lungs they may be more buoyant, thereby affecting the accuracy of the results. With the residual lung volume determined, the system 102 computes the overall body density. Also, the system 102 measures the dry weight of the one or more users. The dry weight refers to the weight determined on land of the subject 312. Further, the system 102 measures wet weight of the subject 312. The wet weight refers to the weight determined in e.g., water of the subject 312. Further, the system 102 determines density of the e.g., water. The density of water generally changes slightly with temperature and salinity. Furthermore, the system 102 determines the body volume based on the determined wet weight and the density of water. The body volume can be computed by dividing the wet weight of the subject 312 by the density of the water. Further, the system 102 computes the overall body density based on the determined dry weight and the determined body volume. The overall body density is computed by dividing dry weight by the body volume. Further, the system 102 computes the one or more user's fat mass and fat-free mass, by using a correlation to correlate the computed body density to the fat mass fraction. Additionally, the system 102 monitors the subject 312 with e.g., an infrared (IR)/false-color camera (i.e., image capturing device 306) to view the subject 312 and the container 116. Additionally, the system 102 collects load-vs-time data from the weighing device 110 and enables displaying the time-series data on the display 304, such that the one or more users or the subject 312 can accept/reject/check the time-series data before generating a final report.

Furthermore, the system 102 displays on the display 304, the fat mass and fat-free mass composition using a graphical user interface (GUI) screen of the user device 106 corresponding to the subject 312. The system 102 comprises the user device 106. For example, the user device 106 can also be connected to any of the one or more electronic devices. The user device 106 outputs the fat mass and fat-free mass composition on the GUI screen of the user device 106.

Additionally, the system 102 includes remote assistance, a levelling screws/mechanism, use of limit switches to detect level/attitude/contact, a breath analyser to measure residual lung volume, a speaker tone to help user, and an alarm triggered on the completion of the measurement. The system 102 automates the measurement process rendering the system user-friendly while also enabling the measurement of one or more users in solitude, without the need to rely on a measurement technician.

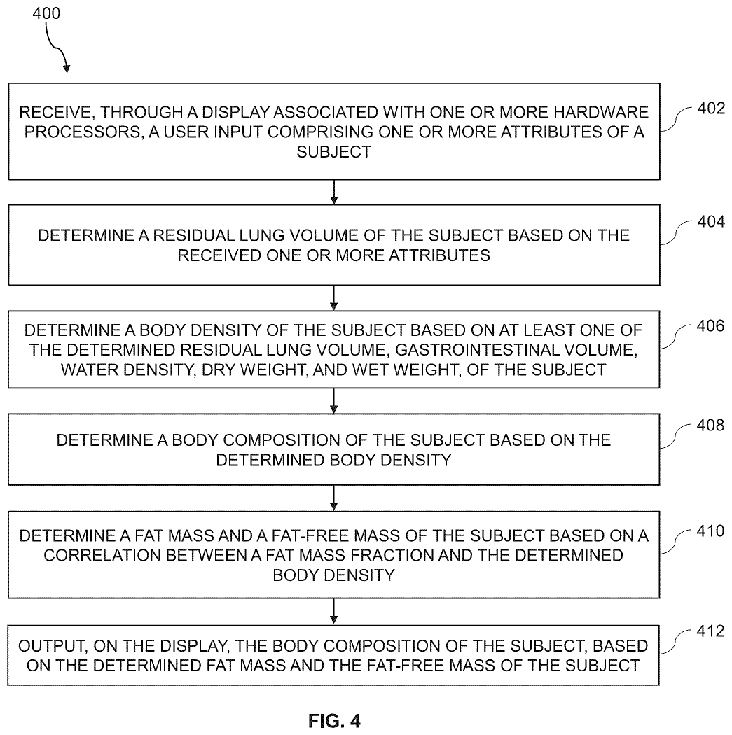

FIG. 4 illustrates a flow chart depicting a method 400 for determining the body composition in the hydrostatic environment, according to an example embodiment of the present disclosure.

At block 402, the method 400 may include receiving, by the one or more hardware processors 118 associated with the system 102, through the display 304 associated with the one or more hardware processors 118, the user input comprising one or more attributes of the subject 312.

At block 404, the method 400 may include determining, by the one or more hardware processors 118, at least one of the residual lung volume of the subject 312 based on the received one or more attributes.

At block 406, the method 400 may include determining, by the one or more hardware processors 118, the body density of the subject 312 based on at least one of the determined residual lung volume (as shown in Equation (1)), the gastrointestinal volume (as shown in Equation (3)), the water density (as shown in Equation (2)), the dry weight, and the wet weight, of the subject 312.

At block 408, the method 400 may include determining, by the one or more hardware processors 118, the body composition of the subject 312 based on the determined body density.

At block 410, the method 400 may include determining, by the one or more hardware processors 118, the fat mass and a fat-free mass of the subject 312 based on the correlation between the fat mass fraction (as shown in Equation 4) and the determined body density.

At block 412, the method 400 may include outputting, by the one or more hardware processors 118, on the display 304, the body composition of the subject 312, based on the determined fat mass and the fat-free mass of the subject 312.

The method 400 may be implemented in any suitable hardware, software, firmware, or combination thereof. The order in which the method 400 is described is not intended to be construed as a limitation, and any number of the described method blocks may be combined or otherwise performed in any order to implement the method 400 or an alternate method. Additionally, individual blocks may be deleted from the method 400 without departing from the spirit and scope of the present disclosure described herein. Furthermore, the method 400 may be implemented in any suitable hardware, software, firmware, or a combination thereof, that exists in the related art or that is later developed. The method 400 describes, without limitation, the implementation of the system 102. A person of skill in the art will understand that method 400 may be modified appropriately for implementation in various manners without departing from the scope and spirit of the disclosure.

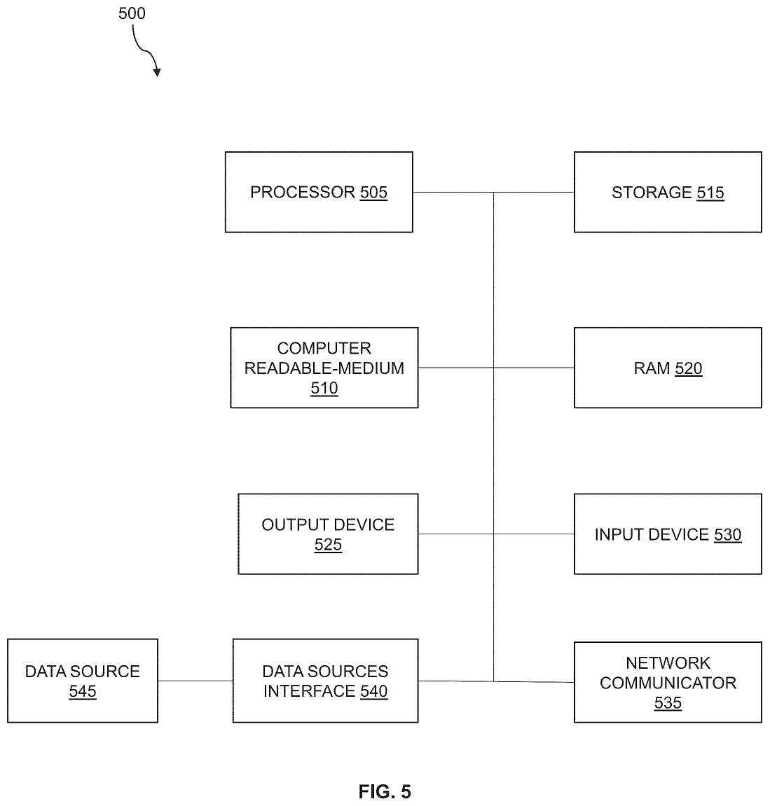

FIG. 5 illustrates an exemplary block diagram representation of a hardware platform 500 for implementation of the disclosed system 102, according to an example embodiment of the present disclosure. For the sake of brevity, the construction, and operational features of the system 102 which are explained in detail above are not explained in detail herein. Particularly, computing machines such as but not limited to internal/external server clusters, quantum computers, desktops, laptops, smartphones, tablets, and wearables which may be used to execute the system 102 or may include the structure of the hardware platform 500. As illustrated, the hardware platform 500 may include additional components not shown, and some of the components described may be removed and/or modified. For example, a computer system with multiple GPUs may be located on external-cloud platforms including Amazon Web Services, or internal corporate cloud computing clusters, or organizational computing resources.

The hardware platform 500 may be a computer system such as the system 102 that may be used with the embodiments described herein. The computer system may represent a computational platform that includes components that may be in a server or another computer system. The computer system may execute, by the processor 505 (e.g., single, or multiple processors) or other hardware processing circuits, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the processor 505 that executes software instructions or code stored on a non-transitory computer-readable storage medium 510 to perform methods of the present disclosure. The software code includes, for example, instructions to gather data and analyze the data. For example, the plurality of modules 122 includes the input receiving module 206, the medical attribute determining module 208, the density determining module 210, the composition determining module 212, the mass determining module 214, the data displaying module 216, the load data recording module 218, and the report generating module 220.

The instructions on the computer-readable storage medium 510 are read and stored the instructions in storage 515 or random-access memory (RAM). The storage 515 may provide a space for keeping static data where at least some instructions could be stored for later execution. The stored instructions may be further compiled to generate other representations of the instructions and dynamically stored in the RAM such as RAM 520. The processor 505 may read instructions from the RAM 520 and perform actions as instructed.

The computer system may further include the output device 525 to provide at least some of the results of the execution as output including, but not limited to, visual information to users, such as external agents. The output device 525 may include a display on computing devices and virtual reality glasses. For example, the display may be a mobile phone screen or a laptop screen. GUIs and/or text may be presented as an output on the display screen. The computer system may further include an input device 530 to provide a user or another device with mechanisms for entering data and/or otherwise interacting with the computer system. The input device 530 may include, for example, a keyboard, a keypad, a mouse, or a touchscreen. Each of these output devices 525 and input device 530 may be joined by one or more additional peripherals. For example, the output device 525 may be used to display the results such as bot responses by the executable chatbot.

A network communicator 535 may be provided to connect the computer system to a network and in turn to other devices connected to the network including other clients, servers, data stores, and interfaces, for example. A network communicator 535 may include, for example, a network adapter such as a LAN adapter or a wireless adapter. The computer system may include a data sources interface 540 to access the data source 545. The data source 545 may be an information resource. As an example, a database of exceptions and rules may be provided as the data source 545. Moreover, knowledge repositories and curated data may be other examples of the data source 545.

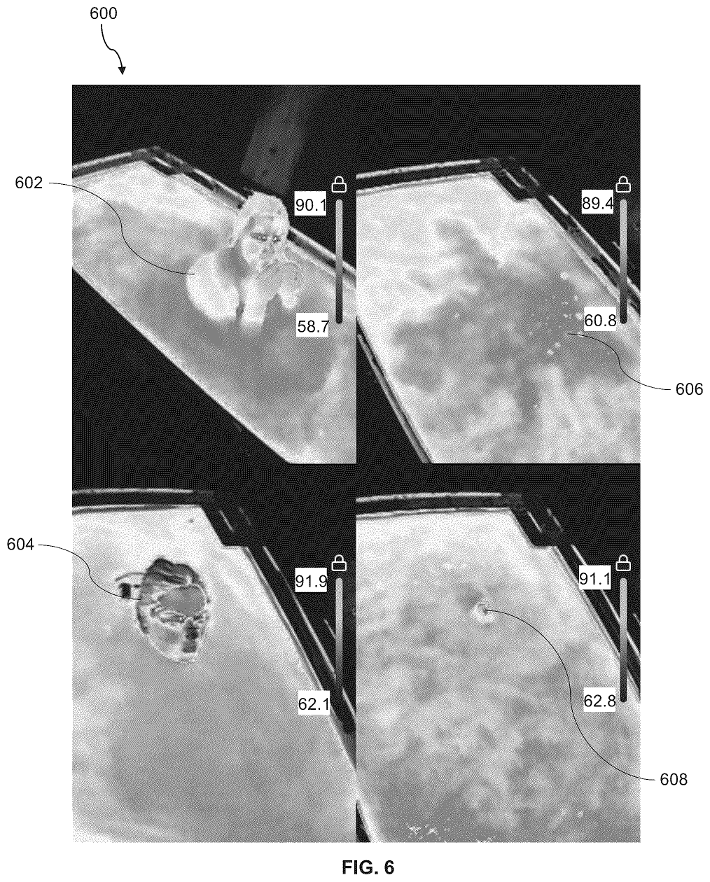

FIG. 6 is an exemplary view 600 depicting an infrared camera image of the subject 312 during a testing procedure, in accordance with an embodiment of the present disclosure. The exemplary view depicts at least one of (a) unsubmerging of the subject 312 (i.e., head and shoulders of the subject 312 are unsubmerged as shown in 602), unsubmerging of the subject 312 (i.e., the head of the subject is unsubmerged as shown in 604), (c) submerging of the subject 312 (i.e., the whole body of the subject 312 is submerged as shown in 606), and (d) unsubmerging of the subject 312 (i.e., nose of the subject 312 is unsubmerged as shown in 608).

FIG. 7 is a graphical representation 700 depicting a typical plot of wet weigh scale output during the testing procedure, such as shown in FIG. 6, in accordance with an embodiment of the present disclosure. The graphical representation 700 depicts a starting position (i.e., between a point “A” 702 and a point “B” 704) that indicates the unsubmerging of the head and shoulders of the subject 312. The graphical representation 700 further depicts a point “C” 706 indicating submerged with partially filled lungs. The graphical representation 700 further depicts a position between a point “D” 708 and a point “E” 710, indicating a steady-state wet weight during empty-lung breath hold. The graphical representation 700 further depicts a finishing position at a point “F” 712 indicating the unsubmerging of the head and shoulders of the subject 312. The graphical representation 700 further depicts a tolerance band 714 for the steady-state wet weight.

The present invention has the following advantages. The system 102 may be configured to capture and plot the time series data from the load cells such that the one or more users may complete the measurement independently, without the need for a technician to read scale's output with their eye. The system 102 with the display device 304 is configured to generate a plot (as shown in FIG. 7) that clearly depicts the stability of the steady-state wet weight value.

The system 102 utilizes an infrared (IR) camera and two-way radio/intercom system for allowing the technician to provide remote assistance and/or supervise the test (if needed/requested) but in a semi-anonymous format such that the one or more users may have less concern for being self-conscious (i.e., being a viewed by a gym user while in a swim suit). The application (e.g., software) reading the data from at least one of the scale, the IR camera, and the intercom system, is intended to be capable of replacing the technician. An end-user may be required to supply their height and age as “Profile Data” when the end-user logs-in to a website to sign-up and pay for a test.

On the day of the test, the end-user need to enter the testing room/vehicle and complete the dry weight, followed by the wet weight (ideally in solitude). The end-user may wish to inspect the load vs. time plot on the display 304 to make sure the wet weight was completed correctly, but the plot may eliminate the need to trust the technician to read the Scale. The IR camera (and the intercom system) is intended for the technician to be capable of supplying remote support in the semi-anonymous format, in the event that such a need arises. The application (e.g., the software) may ultimately be capable of replacing the technician. Upon completion, the report summarizing the computed body composition (i.e., % fat & % lean) may get delivered to electronic mail address of the end-user, as well as be displayed on the customer dashboard on the website.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising.” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a.” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limited, of the scope of the invention, which is outlined in the following claims

What is claimed is:

1. A system for determining a body composition in a hydrostatic environment, the system comprising:
a weighing device configured to measure at least one of a dry weight, and a wet weight corresponding to a body density of the subject;
a subject load transfer structure, comprising a horizontal platform and a plurality of structural elements coupled to the horizontal platform, configured to receive a load of a subject for determining the wet weight of the subject;
a container, comprising a liquid and the subject load transfer structure within the liquid, for determining the wet weight of the subject in the hydrostatic environment, when the subject is submerged in a pre-defined posture in the liquid;
a container load transfer structure, coupled to the weighing device, configured to transfer the load from the weighing device to the container;
one or more hardware processors communicatively connected to the weighing device and a memory coupled to the one or more hardware processors, wherein the memory comprises a plurality of modules in form of programmable instructions executable by the one or more hardware processors, wherein the plurality of modules comprises:
an input receiving module configured to receive, through a display associated with the one or more hardware processors, a user input comprising one or more attributes of the subject;
a medical attribute determining module configured to determine a residual lung volume of the subject based on the received one or more attributes;
a density determining module configured to determine the body density of the subject based on at least one of the determined residual lung volume, gastrointestinal volume, water density, the dry weight, and the wet weight, of the subject;
a composition determining module configured to determine the body composition of the subject based on the determined body density;
a mass determining module configured to determine a fat mass and a fat-free mass of the subject based on a correlation between a fat mass fraction and the determined body density; and
a data displaying module configured to output on the display the body composition of the subject, based on the determined fat mass and the fat-free mass of the subject.

2. The system of claim 1, wherein the plurality of modules further comprises:
a load data recoding module configured to record, for a pre-defined period, load data corresponding to the load of the subject, from the weighing device;
a report generating module configured to generate one or more reports based on the recorded, for a pre-defined period, load data; and
the data displaying module configured to display on the display, the generated one or more reports for inspection, by at least one of the subject and one or more users, of a steady-state value of the body composition.

3. The system of claim 1, further comprising:
an image capturing device configured to monitor the subject during the measurement of the wet weight, using one or more image processing based artificial intelligence (AI) techniques.

4. The system of claim 3, wherein monitoring the subject comprises determining if the subject is submerged in the pre-defined posture in the liquid.

5. The system of claim 1, further comprising:
a speaker device configured to output an audio corresponding to at least one of a beginning of the measurement of at least one of the dry weight and the wet weight, a completion of the measurement of at least one of the dry weight and the wet weight, a formation instruction of the pre-defined posture, and a breath hold instruction for an empty-lung breath hold of the subject; and
a microphone configured to receive an utterance from at least one of the subject and the one or more users, for a remote support corresponding to the measurement.

6. The system of claim 5, wherein the wet weight of the subject is measured based on the empty-lung breath hold of the subject to reduce the lungs of the subject to the residual lung volume.

7. The system of claim 1, further comprising:
a levelling member, comprising one or more limit switches and coupled to the container, configured to detect at least one of a level of the container, an attitude of the container, and a contact status of the container.

8. The system of claim 1, wherein the plurality of structural elements is coupled to the weighing device through a weighing device interface plate.

9. The system of claim 8, wherein the weighing device interface plate is configured to transfer the load of the subject from the plurality of structural elements to the weighing device.

10. The system of claim 1, wherein the one or more attributes comprises at least one of a height and an age of the subject.

11. A method for determining a body composition in a hydrostatic environment, the method comprising:
receiving, by one or more hardware processors associated with a system, through a display associated with the one or more hardware processors, a user input comprising one or more attributes of a subject;
determining, by the one or more hardware processors, a residual lung volume of the subject based on the received one or more attributes;
determining, by the one or more hardware processors, a body density of the subject based on at least one of the determined residual lung volume, gastrointestinal volume, water density, a dry weight, and a wet weight, of the subject;
determining, by the one or more hardware processors, the body composition of the subject based on the determine body density;
determining, by the one or more hardware processors, a fat mass and a fat-free mass of the subject based on a correlation between a fat mass fraction and the determined body density; and
outputting, by the one or more hardware processor, on the display, the body composition of the subject, based on the determined fat mass and the fat-free mass of the subject.

12. The method of claim 11, further comprising:
recording, by the one or more hardware processors, for a pre-defined period, load data corresponding to the load of the subject, from the weighing device;
generating, by the one or more hardware processors, one or more reports based on the recorded, for a pre-defined period, load data; and
displaying, by the one or more hardware processors, on the display, the generated one or more reports for inspection, by at least one of the subject and one or more users of a steady-state value of the body composition.

13. The method of claim 11, further comprising:
monitoring, by the one or more hardware processors, through an image capturing device, the subject during the measurement of the wet weight, using one or more image processing based artificial intelligence (AI) techniques.

14. The method of claim 13, wherein monitoring the subject comprises determining, by the one or more hardware processors, if the subject is submerged in the pre-defined posture in the liquid.

15. The method of claim 11, further comprising:
outputting, by the one or more hardware processors, through a speaker device, an audio corresponding to at least one of a beginning of the measurement of at least one of the dry weight and the wet weight, a completion of the measurement of at least one of the dry weight and the wet weight, a formation instruction of the pre-defined posture, and a breath hold instruction for an empty-lung breath hold of the subject; and
receiving, by the one or more hardware processors, through a microphone, an utterance from at least one of the subject and the one or more users, for a remote support corresponding to the measurement.

16. The method of claim 15, wherein the wet weight of the subject is measured based on the empty-lung breath hold of the subject to reduce the lungs of the subject to the residual lung volume.

17. The method of claim 11, further comprising:
detecting, by the one or more hardware processors, through a levelling member, at least one of a level of the container, an attitude of the container, and a contact status of the container.

18. The method of claim 11, wherein the plurality of structural elements is coupled to the weighing device through a weighing device interface plate, and wherein the weighing device interface plate is configured to transfer the load of the subject from the plurality of structural elements to the weighing device.

19. The method of claim 11, wherein the one or more attributes comprises at least one of a height and an age of the subject.

20. A non-transitory computer-readable storage medium having programmable instructions stored therein, that when executed by one or more hardware processors, cause the one or more hardware processors to:
receive, through a display associated with the one or more hardware processors, a user input comprising one or more attributes of a subject;
determine a residual lung volume of the subject based on the received one or more attributes;
determine a body density of the subject based on at least one of the determined residual lung volume, gastrointestinal volume, water density, a dry weight, and a wet weight, of the subject;
determine the body composition of the subject based on the determine body density;
determine a fat mass and a fat-free mass of the subject based on a correlation between a fat mass fraction and the determined body density; and
output, on the display, the body composition of the subject, based on the determined fat mass and the fat-free mass of the subject.
